(19) United States
(12) Patent Application Publication
(10) Pub. No.: US 2023/0414376 A1
(43) Pub. Date: Dec. 28, 2023

(54) ORTHOPEDIC PROSTHESIS, PARTICULARLY INTERSOMATIC CAGE

(71) Applicant: SPS S.r.l., Scarmagno (Torino) (IT)

(72) Inventor: Valerio VALLANA, Torino (IT)

(21) Appl. No.: 18/249,809

(22) PCT Filed: Oct. 20, 2021
(86) PCT No.: PCT/IB2021/059658

§ 371 Date: Apr. 20, 2023

(30) Foreign Application Priority Data
Oct. 23, 2020 (IT) 102020000025192

(51) Int. Cl. A61F 2/44; A61N 5/06

(52) U.S. Cl. CPC A61F 2/447; A61N 5/0601; A61N 5/0624; A61F 2002/30668; A61N 2005/0661; A61N 2005/0626; A61N 2005/0652

(57) ABSTRACT

An orthopedic prosthesis, particularly an intersomatic cage for vertebral stabilization, incorporating a plurality of light emitting diodes (LEDs) with controlled emission of ultraviolet rays.

### FIELD OF THE INVENTION

The present invention generally relates to the orthopaedic prosthesis such as, by way of non-limiting example, the intersomatic cages for vertebral stabilisation designed to be inserted between two contiguous vertebrae so as to space them apart and therefore keep them at a mutual distance such to restore the intervertebral space, creating the decompression of the nerve roots and the acceleration of the intersomatic fusion.

### STATE OF THE ART

Intersomatic cages thus made typically comprise a body provided with external anchoring formations and with a nose protruding from the front end of the body and designed to be inserted between two contiguous vertebrae during the surgical insertion of the intersomatic cage.

Following implantation of the cage, the patient should normally be treated from a pharmaceutical point of view in order to avoid the onset of infections as much as possible. Typically, treatment consists in the administration of antibiotics, which however reveal two types of problems: the risk of the patient developing a resistance which reduces or even nullifies antibiotic efficacy, and the onset of possible adverse reactions that can develop even more serious infections.

Similar problems occur with orthopaedic prostheses in general.

It is therefore desirable to avoid the use of antibiotics in postoperative hospitalisation, replacing them with safer and at the same time not less effective alternative measures.

Documents US 2012/109304, US 2005/175658 and WO 2006/041670 disclose orthopaedic prostheses incorporating leds with controlled emission of ultraviolet rays. The efficacy of UV radiations in terms of sterilising, disinfecting and antibacterial properties is per se known: for example, the United States Patent application US 2006/0183987 describes a venous implant for cancer phototherapy consisting of a ring of UV rays emitter leds which can be connected to an electrical power source by means of a cable.

### SUMMARY OF THE INVENTION

The object of the present invention is to improve the functional efficacy of an intersomatic cage provided with leds with controlled emission of ultraviolet rays and such object is achieved thanks to the characteristics set out in the claims below.

Thanks to the invention, the intersomatic cage allows to provide, following the implantation thereof, a more effective selective treatment of the orthopaedic region and of the vertebral region in this case. In particular, the miniaturised leds are distributed superficially on the faces of the cage, specifically applied to the anchoring indentations, and they are connected to an activation circuit, also miniaturised and integrated in the cage, power-supplied by an external and remote electrical source. The power supply is conveniently wireless, carried out for example by induction or by means of radio frequency (RF) power waves.

### BRIEF DESCRIPTION OF THE DRAWINGS

The invention will now be described in detail with reference to the attached drawings, provided purely by way of non-limiting example, wherein:

FIG. 1 is a schematic perspective view of an orthopaedic prosthesis according to the invention,

FIG. 2 is a diagram exemplifying the power supply of the prosthesis according to the invention.

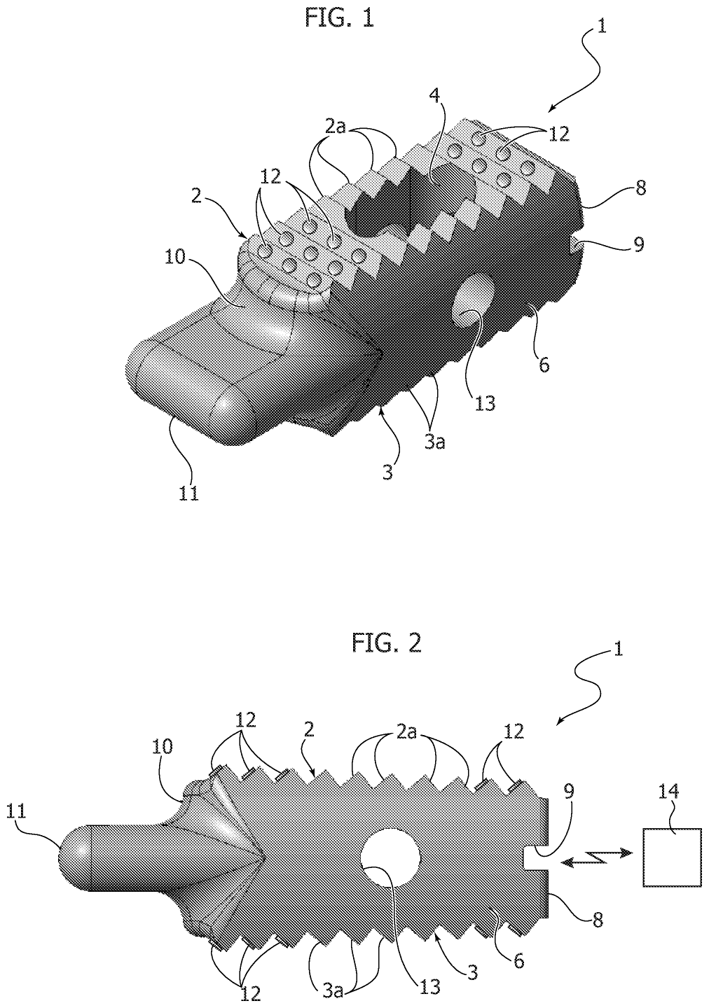

### DETAILED DESCRIPTION OF THE INVENTION

The embodiment described herein refers to the particular case of an intersomatic cage for vertebral stabilisation. It should be observed that the invention is generally equally advantageously applicable to any orthopaedic prosthesis.

With reference to the figures, the intersomatic cage according to the invention consists of a generally prismatic-shaped and more precisely parallelepiped-shaped monolithic body 1, having an upper face 2 and a lower face 3 (with reference to the implanted position of the cage in the intervertebral space of a subject with an upright vertebral column), both formed with respective anchoring formations consisting of parallel indentations 2a, 3a.

A slot-shaped through opening 4 vertically traverses the body 1 between the upper 2 and lower 3 faces, and a circular through hole 5 extends between the lateral walls 6, 7 of the body 1.

As observable in FIG. 4, the body 1 has a height, measured between the upper 2 and lower 3 faces, which decreases slightly toward the rear end thereof, indicated with 8 and in which a recess 9 for the introduction of an instrument for the surgical insertion of the cage into the intervertebral space. This instrument, not illustrated, is configured so as to engage axially and torsionally with the recess 9 so as to be able to rotate the body 1 alternately in a clockwise and anti-clockwise direction while being simultaneously pushed.

A nose 11—which has the function of paving the way by dilating the intervertebral space during the process for inserting the intersomatic cage—protrudes from the front end of the body 1, indicated with reference numeral 10. The nose 11 extends parallel to the upper 2 and lower 3 faces of the body 1 and it has a substantially quadrangular shape with a constant cross-section and rounded edges. More precisely, the nose 11 extends on the extension of a median longitudinal plane of the body 1 and therefore protrudes from a median area of the front end 10, connecting to such median area through curved surfaces, preferably having variable radiuses, clearly visible in the figures.

According to the distinctive characteristic of the invention, the body 1 of the cage incorporates a plurality of leds 12 with controlled emission of ultraviolet rays.

The miniaturised leds 12 are distributed superficially at least on the faces 2, 3 of the cage and more particularly they are applied on at least part of the anchoring formations consisting of the parallel indentations 2a, 3a.

As clearly observable in the drawings, the leds 12 are advantageously fixed on the front inclined walls (with reference to the direction of insertion of the cage in the intervertebral space) of at least part of the indentations 2a, 2b. In this manner, in the implanted condition of the cage, the leds will be directly in contact with the vertebral regions.

The leds 12 are operatively connected to an activation or energisation circuit 13, which is also miniaturised and preferably, though not necessarily, integrated in the cage body 1. The activation circuit 13 is power-supplied by an internal or external electrical source: in the latter case, power is conveniently transmitted in wireless mode and is carried out for example by induction or by means of a remote emitter 14 of radio frequency (RF) power waves.

Alternatively, the activation circuit 13 may be provided for outside the cage, and the electrical power source thereof could also consist of a battery housed in the body 1 or arranged externally.

The leds 12 with controlled emission of ultraviolet rays may also be provided for on other parts of the body 1, for example also on the nose 11.

Obviously, the construction details and the embodiments may widely vary with respect to what has been described and illustrated, without departing from the scope of protection of the invention as described in the claims that follow.

1. An orthopedic prosthesis in the form of an intersomatic cage for vertebral stabilization, comprising:
a generally parallelepiped body having two respectively upper and lower opposite faces, with reference to an implanted position of the intersomatic cage in an intervertebral space of a subject with an upright vertebral column, provided with respective anchoring formations,
said orthopedic prosthesis incorporating a plurality of light emitting diodes (LEDs) with controlled emission of ultraviolet rays,
wherein the LEDs with controlled emission of ultraviolet rays are arranged at least on the upper and lower faces of the body of the intersomatic cage and are applied on at least part of said anchoring formations.

2. The orthopedic prosthesis according to claim 1, wherein the LEDs with controlled emission of ultraviolet rays are applied on front surfaces, with reference to a direction of insertion of the intersomatic cage in the intervertebral space, of said anchoring formations.

3. The orthopedic prosthesis according to claim 1, wherein the LEDs with controlled emission of ultraviolet rays are operatively connected to an activation circuit.

4. The orthopedic prosthesis according to claim 3, wherein the activation circuit is integrated in the body of the orthopedic prosthesis.

5. The orthopedic prosthesis according to claim 3, wherein the activation circuit is supplied by a remote electrical source.

6. The orthopedic prosthesis according to claim 3, wherein the activation circuit is power-supplied in wireless mode.

7. The orthopedic prosthesis according to claim 3, wherein the activation circuit is power-supplied by a battery.

8. The orthopedic prosthesis according to claim 7, wherein the intersomatic cage comprises a nose protruding from a front end of the body and adapted to be inserted between two contiguous vertebrae to space them apart them during surgical insertion of the cage,
wherein the nose extends parallel to said upper and lower faces and is substantially quadrangular-shaped with constant cross-section and rounded edges, and
wherein the LEDs with controlled emission of ultraviolet rays are arranged on said nose.
